SEQUENCE LISTING (15 sequences)

SEQ ID NO: 1 - protein, 566 aa, Artificial Sequence
  Synthetic Construct
MKARLLVLLCALAATDADTICIGYHANNSTDTVDTVLEKNVTVTHSVNLLEDSHNGKLCKLKGIAPLQLGKCNIAGWLLGNPECESLISKESWSYIVETPNSENGTCYPGDFIDYEELREQLSSVSSFERFEIFPKESSWPNHNTTKGVTAACSHAGKSSFYRNLLWLTKKGGSYPKLSKSYVNNKGKEVLVLWGVHHPSTSTDQQSLYQNENAYVSVVSSNYNRRFTPEIAERPKVRGQAGRMNYYWTLLEPGDTIIFEATGNLIAPWYAFALSRGSGSGIITSNASMHECNTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKSQLRNNAKEIGNGCFEFYHKCDNECMESVKNGTYDYPKYSEESKLNREKIDGVKLESMGVYQILAIYSTVASSLVLLVSLGAISFWMCSNGSLQCRICI

SEQ ID NO: 2 - DNA, 1710 bp, Artificial Sequence
  Synthetic Construct
aagcttatgaaggccagactgctggtcctgctgtgtgctctcgctgccaccgacgccgataccatctgcatcggctaccatgccaataactccaccgacaccgtggacaccgtgctggagaagaacgtcacagtcacccactccgtgaacctcctcgaggactcccacaatggcaagctgtgcaagctgaagggaatcgcccccctgcagctgggcaagtgcaacatcgccggatggctgctcggaaatcccgagtgtgagtccctgatcagcaaggagtcctggagctatatcgtggaaacccccaacagcgagaatggcacctgctaccctggagacttcattgattacgaagaactgagggagcaactctccagcgtgagcagcttcgagaggttcgaaatcttccccaaggagagctcctggcccaaccacaacaccaccaagggagtgacagccgcttgctcccacgctggaaagtcctccttctacagaaacctgctgtggctcaccaagaagggcgggagctaccccaagctgtccaagtcctacgtgaacaacaagggcaaggaggtgctggtcctgtggggcgtccatcaccccagcaccagcaccgatcaacagtccctgtaccagaacgagaacgcctacgtgagcgtcgtgagcagcaactacaacaggaggtttacccctgagatcgccgaaagacccaaggtgagaggccaggctggcagaatgaattactactggaccctgctggagcctggcgacaccatcatcttcgaggccacaggcaacctgatcgccccctggtatgcctttgccctcagcagaggctccgggagcggcatcatcacaagcaatgccagcatgcacgagtgcaacaccaaatgtcagaccccccaaggcgctattaacagcagcctgcctttccagaacatccaccctgtgaccatcggcgaatgtcccaagtacgtcaggagcaccaagctcagaatggtgaccggcctgaggaatatccccagcattcaaagcaggggcctgtttggagccatcgccggattcatcgagggaggctggaccggaatgatcgacggctggtatggctaccatcaccagaacgaacagggctccggctatgccgccgaccagaaaagcacccagaacgccatcaatggcatcaccaataaagtcaattccgtcattgagaagatgaatacccagttcaccgccgtgggcaaggagtttaataatctggagaagagaatggagaatctgaacaagaaggtggacgacggcttcctcgacatttggacatacaacgccgagctcctggtcctgctggagaacgaaaggacactggacttccacgactccaacgtgaagaacctctacgagaaggtgaagagccagctcaggaacaacgccaaggagattggaaacggctgctttgagttctaccacaagtgcgacaacgaatgcatggagagcgtgaagaacggaacctatgactaccccaagtactccgaagagagcaaactgaacagggagaagatcgatggcgtgaagctcgaatccatgggcgtgtaccagatcctggccatctactccacagtcgccagcagcctggtgctgctggtgagcctgggcgccatctccttctggatgtgcagcaacgggagcctgcagtgtaggatctgcatctgatag

SEQ ID NO: 3 - protein, 565 aa, Artificial Sequence
  Synthetic Construct
MKARLLVLLCALAATDADTICIGYHANNSTDTVDTVLEKNVTVTHSVNLLEDSHNGKLCKLKGIAPLQLGKCNIAGWLLGNPECESLLSARSWSYIVETPNSENGTCYPGDFIDYEELREQLSSVSSFERFEIFPKESSWPNHTVTGVTAACSHAGKSSFYRNLLWLTKKGGSYPKLSKSYVNNKGKEVLVLWGVHHPSTSTDQQSLYQNENAYVSVVSSNYNRRFTPEIAERPKVRGQAGRMNYYWTLLEPGDTIIFEATGNLIAPWYAFALSRGSGSGIITSNASMHECNTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKSQLRNNAKEIGNGCFEFYHKCDNECMESVKNGTYDYPKYSEESKLNREKIDGVKLESMGVYQILAIYSTVASSLVLLVSLGAISFWMCSNGSLQCRICI

SEQ ID NO: 4 - DNA, 1707 bp, Artificial Sequence
  Synthetic Construct
aagcttatgaaggccagactgctggtcctgctgtgtgctctcgctgccaccgacgccgataccatctgcatcggctaccatgccaataactccaccgacaccgtggacaccgtgctggagaagaacgtcacagtcacccactccgtgaacctcctcgaggactcccacaatggcaagctgtgcaagctgaagggaatcgcccccctgcagctgggcaagtgcaacatcgccggatggctgctcggaaatcccgagtgtgagtccctgctgagcgctaggtcctggagctatatcgtggaaacccccaacagcgagaatggcacctgctaccctggagacttcattgattacgaagaactgagggagcaactctccagcgtgagcagcttcgagaggttcgaaatcttccccaaggagagctcctggcccaaccacaccgtgaccggagtgacagccgcttgctcccacgctggaaagtcctccttctacagaaacctgctgtggctcaccaagaagggcgggagctaccccaagctgtccaagtcctacgtgaacaacaagggcaaggaggtgctggtcctgtggggcgtccatcaccccagcaccagcaccgatcaacagtccctgtaccagaacgagaacgcctacgtgagcgtcgtgagcagcaactacaacaggaggtttacccctgagatcgccgaaagacccaaggtgagaggccaggctggcagaatgaattactactggaccctgctggagcctggcgacaccatcatcttcgaggccacaggcaacctgatcgccccctggtatgcctttgccctcagcagaggctccgggagcggcatcatcacaagcaatgccagcatgcacgagtgcaacaccaaatgtcagaccccccaaggcgctattaacagcagcctgcctttccagaacatccaccctgtgaccatcggcgaatgtcccaagtacgtcaggagcaccaagctcagaatggtgaccggcctgaggaatatccccagcattcaaagcaggggcctgtttggagccatcgccggattcatcgagggaggctggaccggaatgatcgacggctggtatggctaccatcaccagaacgaacagggctccggctatgccgccgaccagaaaagcacccagaacgccatcaatggcatcaccaataaagtcaattccgtcattgagaagatgaatacccagttcaccgccgtgggcaaggagtttaataatctggagaagagaatggagaatctgaacaagaaggtggacgacggcttcctcgacatttggacatacaacgccgagctcctggtcctgctggagaacgaaaggacactggacttccacgactccaacgtgaagaacctctacgagaaggtgaagagccagctcaggaacaacgccaaggagattggaaacggctgctttgagttctaccacaagtgcgacaacgaatgcatggagagcgtgaagaacggaacctatgactaccccaagtactccgaagagagcaaactgaacagggagaagatcgatggcgtgaagctcgaatccatgggcgtgtaccagatcctggccatctactccacagtcgccagcagcctggtgctgctggtgagcctgggcgccatctccttctggatgtgcagcaacgggagcctgcagtgtaggatctgcatctgatag

SEQ ID NO: 5 - protein, 565 aa, Artificial Sequence
  Synthetic Construct
MKARLLVLLCALAATDADTICIGYHANNSTDTVDTVLEKNVTVTHSVNLLEDSHNGKLCKLKGIAPLQLGKCNIAGWLLGNPECESLISKESWSYIVETPNSENGTCYPGDFIDYEELREQLSSVSSFERFEIFPKESSWPNHTVTGVTAACSHAGKSSFYRNLLWLTKKGGSYPKLSKSYVNNKGKEVLVLWGVHHPSTSTDQQSLYQNENAYVSVVSSNYNRRFTPEIAERPKVRGQAGRMNYYWTLLEPGDTIIFEATGNLIAPWYAFALSRGSGSGIITSNASMHECNTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKSQLRNNAKEIGNGCFEFYHKCDNECMESVKNGTYDYPKYSEESKLNREKIDGVKLESMGVYQILAIYSTVASSLVLLVSLGAISFWMCSNGSLQCRICI

SEQ ID NO: 6 - DNA, 1707 bp, Artificial Sequence
  Synthetic Construct
aagcttatgaaggccagactgctggtcctgctgtgtgctctcgctgccaccgacgccgataccatctgcatcggctaccatgccaataactccaccgacaccgtggacaccgtgctggagaagaacgtcacagtcacccactccgtgaacctcctcgaggactcccacaatggcaagctgtgcaagctgaagggaatcgcccccctgcagctgggcaagtgcaacatcgccggatggctgctcggaaatcccgagtgtgagtccctgatcagcaaggagtcctggagctatatcgtggaaacccccaacagcgagaatggcacctgctaccctggagacttcattgattacgaagaactgagggagcaactctccagcgtgagcagcttcgagaggttcgaaatcttccccaaggagagctcctggcccaaccacaccgtgaccggagtgacagccgcttgctcccacgctggaaagtcctccttctacagaaacctgctgtggctcaccaagaagggcgggagctaccccaagctgtccaagtcctacgtgaacaacaagggcaaggaggtgctggtcctgtggggcgtccatcaccccagcaccagcaccgatcaacagtccctgtaccagaacgagaacgcctacgtgagcgtcgtgagcagcaactacaacaggaggtttacccctgagatcgccgaaagacccaaggtgagaggccaggctggcagaatgaattactactggaccctgctggagcctggcgacaccatcatcttcgaggccacaggcaacctgatcgccccctggtatgcctttgccctcagcagaggctccgggagcggcatcatcacaagcaatgccagcatgcacgagtgcaacaccaaatgtcagaccccccaaggcgctattaacagcagcctgcctttccagaacatccaccctgtgaccatcggcgaatgtcccaagtacgtcaggagcaccaagctcagaatggtgaccggcctgaggaatatccccagcattcaaagcaggggcctgtttggagccatcgccggattcatcgagggaggctggaccggaatgatcgacggctggtatggctaccatcaccagaacgaacagggctccggctatgccgccgaccagaaaagcacccagaacgccatcaatggcatcaccaataaagtcaattccgtcattgagaagatgaatacccagttcaccgccgtgggcaaggagtttaataatctggagaagagaatggagaatctgaacaagaaggtggacgacggcttcctcgacatttggacatacaacgccgagctcctggtcctgctggagaacgaaaggacactggacttccacgactccaacgtgaagaacctctacgagaaggtgaagagccagctcaggaacaacgccaaggagattggaaacggctgctttgagttctaccacaagtgcgacaacgaatgcatggagagcgtgaagaacggaacctatgactaccccaagtactccgaagagagcaaactgaacagggagaagatcgatggcgtgaagctcgaatccatgggcgtgtaccagatcctggccatctactccacagtcgccagcagcctggtgctgctggtgagcctgggcgccatctccttctggatgtgcagcaacgggagcctgcagtgtaggatctgcatctgatag

SEQ ID NO: 7 - protein, 449 aa, Artificial Sequence
  Synthetic Construct
MNPNKKIITIGSICMVTGMVSLMLQIGNLISIWVSHSIHTGNQHKAEPISNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 8 - DNA, 1350 bp, Artificial Sequence
  Synthetic Construct
atgaatcctaataagaagatcatcacaatcggaagcatctgcatggtgacaggaatggtgagcctgatgctgcagatcggaaatctgatcagcatctgggtgagccacagcatccacacaggaaatcagcacaaggccgagcctatcagcaatacaaattttctgacagagaaggccgtggccagcgtgaagctggccggaaatagcagcctgtgccctatcaatggatgggccgtgtacagcaaggataatagcatcagaatcggaagcaagggagatgtgtttgtgatcagagagccttttatcagctgcagccacctggagtgcagaacattttttctgacacagggagccctgctgaatgataagcacagcaatggaacagtgaaggatagaagccctcacagaacactgatgagctgccctgtgggagaggcccctagcccttacaatagcagatttgagagcgtggcctggagcgccagcgcctgccacgatggaacaagctggctgacaatcggaatcagcggacctgataatggagccgtggccgtgctgaagtacaatggaatcatcacagatacaatcaagagctggagaaataatatcctgagaacacaggagagcgagtgcgcctgcgtgaatggaagctgctttacagtgatgacagatggacctagcaatggacaggccagccacaagatctttaagatggagaagggaaaggtggtgaagagcgtggagctggatgcccctaattaccactacgaggagtgcagctgctaccctgatgccggagagatcacatgcgtgtgcagagataattggcacggaagcaatagaccttgggtgagctttaatcagaatctggagtaccagatcggatacatctgcagcggagtgtttggagataatcctagacctaatgatggaacaggaagctgcggacctgtgagcagcaatggagcctacggagtgaagggatttagctttaagtacggaaatggagtgtggatcggaagaacaaagagcacaaatagcagaagcggatttgagatgatctgggaccctaatggatggacagagacagatagcagctttagcgtgaagcaggatatcgtggccatcacagattggagcggatacagcggaagctttgtgcagcaccctgagctgacaggactggattgcatcagaccttgcttttgggtggagctgatcagaggaagacctaaggagagcacaatctggacaagcggaagcagcatcagcttttgcggagtgaatagcgatacagtgggatggagctggcctgatggagccgagctgccttttacaatcgataagtga

SEQ ID NO: 9 - protein, 252 aa, Artificial Sequence
  Synthetic Construct
MSLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 10 - DNA, 759 bp, Artificial Sequence
  Synthetic Construct
atgagcctgctgaccgaggtggagacatacgtgctgtccatcatccccagcggccctctgaaggccgagatcgcccagagactggaagatgtgttcgccggcaagaacaccgacctggaagtgctgatggaatggctgaaaaccagacccatcctgagccctctgaccaagggcatcctgggcttcgtgttcaccctgaccgtgcccagcgagagaggcctgcagaggcggagattcgtgcagaacgccctgaacggcaacggcgaccccaacaacatggacaaggccgtgaagctgtacagaaagctgaagcgggagatcaccttccacggcgccaaagagatcagcctgagctacagcgctggcgccctggccagctgcatgggcctgatctacaacagaatgggcgccgtgaccaccgaggtggccttcggcctggtctgcgccacctgcgagcagatcgccgacagccagcacagatcccacagacagatggtcaccaccaccaaccccctgatcagacacgagaacagaatggtgctggcctctaccaccgccaaggccatggaacagatggccggcagcagcgagcaggccgccgaggctatggaagtcgcctctcaggctaggcagatggtccaggccatgagaaccatcggcacccaccccagcagctctgctggcctgaagaacgacctgctggaaaacctgcaggcctaccagaaaagaatgggcgtccagatgcagagattcaagtga

SEQ ID NO: 11 - protein, 555 aa, Influenza Virus
MKANLLVLLCALAAADADTICIGYHANNSTDTVDTVLEKNEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 12 - protein, 566 aa, Influzenza Virus
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 13 - protein, 566 aa, Artificial Sequence
  Synthetic Construct
MKARLLVLLCALAATDADTICIGYHANNSTDTVDTVLEKNVTVTHSVNLLEDSHNGKLCKLKGIAPLQLGKCNIAGWLLGNPECESLLSARSWSYIVETPNSENGTCYPGDFIDYEELREQLSSVSSFERFEIFPKESSWPNHNTTKGVTAACSHAGKSSFYRNLLWLTKKGGSYPKLSKSYVNNKGKEVLVLWGVHHPSTSTDQQSLYQNENAYVSVVSSNYNRRFTPEIAERPKVRGQAGRMNYYWTLLEPGDTIIFEATGNLIAPWYAFALSRGSGSGIITSNASMHECNTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKSQLRNNAKEIGNGCFEFYHKCDNECMESVKNGTYDYPKYSEESKLNREKIDGVKLESMGVYQILAIYSTVASSLVLLVSLGAISFWMCSNGSLQCRICI

SEQ ID NO: 14 - protein, 565 aa, Artificial Sequence
  Synthetic Construct
MEARLLVLLCAFAATNADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRKFTPEIAKRPKVRDQEGRINYYWTLLEPGDTIIFEANGNLIAPRYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 15 - protein, 566 aa, Artificial Sequence
  Synthetic Construct
MEARLLVLLCAFAATNADTICIGYHANNSTDTVDTVLEKNVTVTHSVNLLEDSHNGKLCRLKGIAPLQLGNCSVAGWILGNPECESLFSKESWSYIAETPNPENGTCYPGYFADYEELREQLSSVSSFERFEIFPKESSWPNHTVTKGVTASCSHNGKSSFYRNLLWLTEKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

(19) United States
(12) Patent Application Publication
(10) Pub. No.: US 2020/0237898 A1
(43) Pub. Date: Jul. 30, 2020

(54) VACCINES INTELLIGENTLY PRODUCED BY EPITOPE RECOMBINATION (VIPER) FOR INFLUENZA

(71) Applicant: OREGON HEALTH & SCIENCE UNIVERSITY, Portland, OR (US)

(72) Inventors: Ted Milburn Ross, Watkinsville, GA (US); Terrianne Maiko Wong, Athens, GA (US); Donald Martin Carter, Jr., Athens, GA (US); Corey John Crevar, North Huntington, PA (US)

(73) Assignee: OREGON HEALTH & SCIENCE UNIVERSITY, PORTLAND, OR (US)

(21) Appl. No.: 15/761,779

(22) PCT Filed: Sep. 21, 2016
(86) PCT No.: PCT/US2016/052866

§ 371 Date: Mar. 20, 2018

Related U.S. Application Data
(60) Provisional application No. 62/221,334, filed on Sep. 21, 2015.

(51) Int. Cl. A61K 39/145; C07K 14/005; C12N 7/00; A61K 38/47

(52) U.S. Cl. CPC A61K 39/145; C07K 14/005; C12N 7/00; C12N 2760/16123; C12Y 302/01018; C12N 2760/16122; C12N 2760/16134; A61K 38/47

(57) ABSTRACT

Provided herein are optimized recombinant influenza HA polypeptides that elicit immune responses. Also provided are compositions and kits comprising the optimized HA polypeptides as well as methods of making and using the optimized HA polypeptides.

### CROSS-REFERENCE TO RELATED APPLICATION

This application claims priority to U.S. Provisional Application No. 62/221,334, filed Sep. 21, 2015, which is incorporated by reference herein in its entirety.

### BACKGROUND OF THE INVENTION

Influenza virus is a member of Orthomyxoviridae family. There are three subtypes of influenza viruses, designated influenza A, influenza B, and influenza C. The influenza virus contains a segmented negative-sense RNA genome, which encodes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (PA), and nonstructural protein 2 (NS2). The HA, NA, M1, and M2 are membrane associated, whereas NP, PB1, PB2, PA, and NS2 are nucleocapsid associated proteins. The HA and NA proteins are envelope glycoproteins, responsible for virus attachment and penetration of the viral particles into the cell, and the sources of the major immunodominant epitopes for virus neutralization and protective immunity. Influenza A viruses are classified into subtypes based on antibody responses to HA and NA. These different types of HA and NA form the basis of the H and N distinctions in, for example, H5N1. There are 16 H and 9 N subtypes known, but only H 1, 2 and 3, and N 1 and 2 are commonly found in humans. Both HA and NA proteins are considered the most important components for prophylactic influenza vaccines.

### BRIEF SUMMARY OF THE INVENTION

Provided herein are optimized recombinant influenza HA polypeptides that elicit immune responses. Also provided are compositions and kits comprising the optimized HA polypeptides as well as methods of making and using the optimized HA polypeptides.

### BRIEF DESCRIPTION OF THE DRAWINGS

FIG. 1 shows the amino acid sequence of an exemplary VIPER hemagglutinin (HA) polypeptide, VIPER PV-1 or VIPER1 (SEQ ID NO: 1).

FIG. 2 shows the nucleic acid sequence of an exemplary VIPER hemagglutinin (HA) polypeptide, VIPER PV-1 or VIPER1 (SEQ ID NO:4).

FIG. 3 shows the amino acid sequence of an exemplary VIPER hemagglutinin (HA) polypeptide, VIPER PV-2 or VIPER2 (SEQ ID NO:2).

FIG. 4 shows the nucleic acid sequence of an exemplary VIPER hemagglutinin (HA) polypeptide, VIPER PV-2 or VIPER2 (SEQ ID NO:5).

FIG. 5 shows the amino acid sequence of an exemplary VIPER hemagglutinin (HA) polypeptide, VIPER PV-3 or VIPER3 (SEQ ID NO:3).

FIG. 6 shows the nucleic acid sequence of an exemplary VIPER hemagglutinin (HA) polypeptide, VIPER PV-3 or VIPER3 (SEQ ID NO:6).

FIG. 7 is an image of a Western blot showing detection of VIPER HA polypeptides on the surface of virus-like particles (VLPs) following purification.

FIG. 8 are graphs of HAI assays showing the H1 VIPER HAs demonstrated a broader neutralizing antibody response against both seasonal and pandemic H1N1 viruses

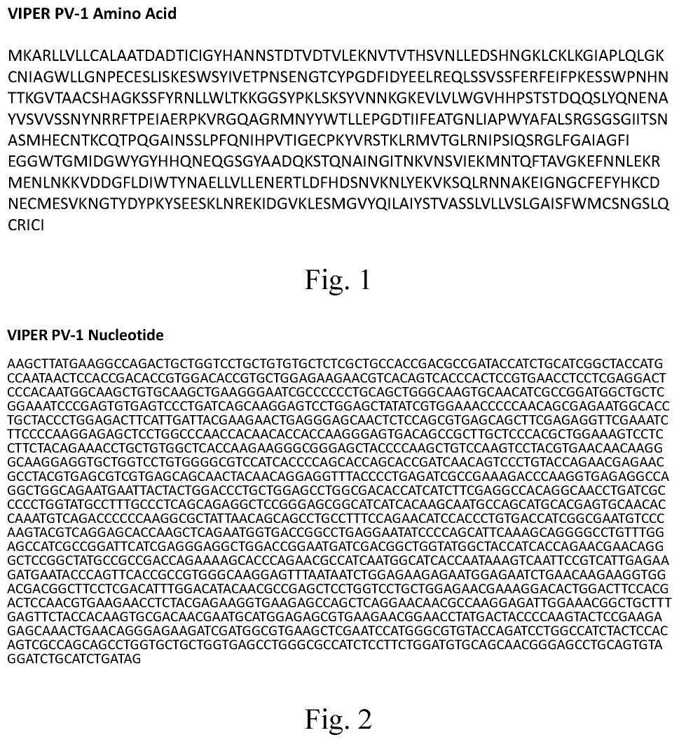

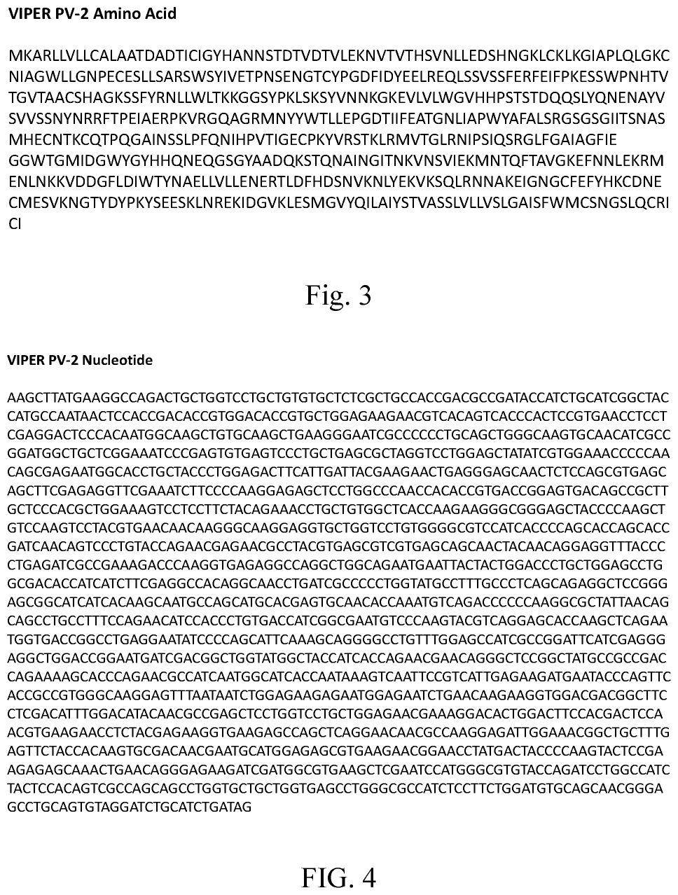

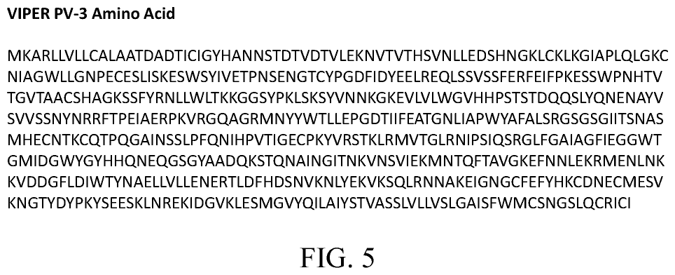

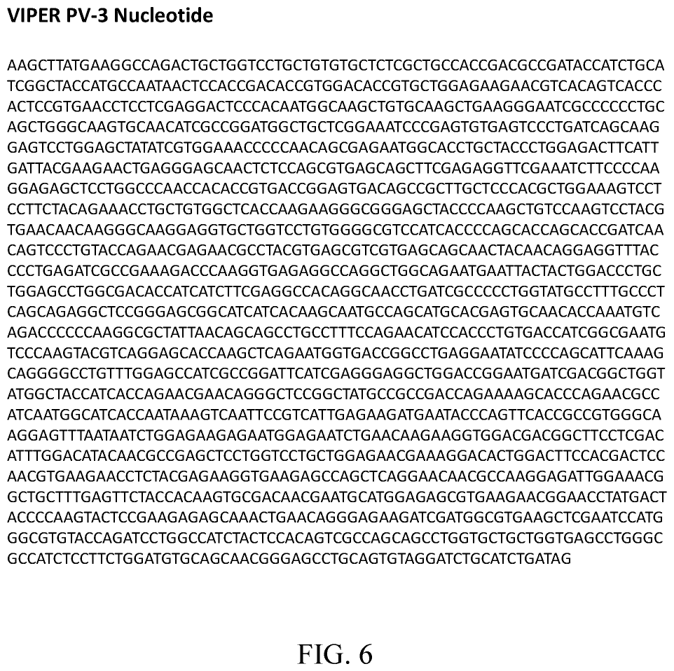

### DETAILED DESCRIPTION OF THE INVENTION

Current vaccination approaches primarily rely on the induction of antibodies recognizing the hemagglutinin (HA) protein. The HA glycoprotein is expressed as a trimeric complex of identical subunits on the surface of influenza virions, and mediates virus attachment and subsequent membrane fusion with target cells (Skehel and Wiley, Annu. Rev. Biochem. 69:531-569 (2000); Bouvier and Palese, Vaccine 12:26, Suppl. 4:D49-53 (2008), which are incorporated by reference herein in their entireties). Individual HA monomers can be further segregated into the membrane distal globular head and membrane proximal stalk domains. The globular head encodes the receptor-binding site (RBS) and the stalk domain encodes the fusion peptide.

Antibodies directed against HA, and more specifically to epitopes in close proximity to the RBS within the globular head region, are elicited following influenza infection or vaccination (Gerhard et al., Nature 290(5808):713-7 (1981); Wilson et al., Virology 458-459:114-24 (2014); Carter, et al., J. Virology, 87(3):1400-10 (2013); Wrammert, et al., Nature, 453(7195):667-71 (2008); Huang, et al., J. Infect. Dis., 209(9): 1354-1361 (2014), which are incorporated by reference herein in their entireties). These antibodies possess potent neutralization capacity through their ability to interfere with viral attachment to target cells and are readily detected using the hemagglutinin inhibition (HAI) assay (Skehel and Wiley, Annu. Rev. Biochem. 69:531-569 (2000); Ohmit, et al., J. Infect. Dis. 204(12): 1879-85 (2011), which are incorporated by reference herein in their entireties). While antibodies with HAI activity can prevent influenza infection, they are largely strain-specific. Accumulation of point mutations within the globular head region of HA, termed antigenic drift, generates viral escape variants and often evasion of pre-existing immunity (Ohmit, et al., J. Infect. Dis. 204(12):1879-85 (2011); Webster, Nature, 296(5853):115-21 (1982); Knossow and Skehel, Immunology 119(1):1-7 (2006), which are incorporated by reference herein in their entireties). Moreover, antigenic drift necessitates frequent reformulation of the seasonal vaccine; a process that is both expensive and time-consuming. Therefore, a need exists for development of vaccines that generate broadly cross-reactive neutralizing antibodies. Such vaccines are provided herein.

Antigenic sites of antibody binding have been identified in the globular head of H1N1, H3N2, and other influenza subtypes that result in antigenic diversity. Using these antigenic regions, specific antigenic sites have been selected to be grafted onto a HA amino acid backbone of either a wild-type or computationally optimized broadly reactive antigen (COBRA) HA protein (Giles and Ross, Vaccine 5:3042-3054 (2011), which is incorporated by reference herein in its entirety). The herein provided HA molecules are, thus, a hybrid or chimeric HA protein comprising an HA backbone and one or more antigenic sites from a different HA molecule. For example, provided are HA molecules with the backbone of a first influenza isolate and the antigenic sites of a second influenza isolate. The isolates can be from the same or different subtypes. The hybrid HA molecules are sometimes referred to herein as VIPER HA molecules or polypeptides. The overall approach generates a unique HA molecule with enhanced abilities to elicit broadly cross-reactive neutralizing antibodies for universal vaccine development. As described herein, VIPER sequences of HA have been generated using H1 HA sequences. However, the methodology is applicable for all subtypes of influenza.

Provided herein are recombinant HA polypeptides comprising antigenic sites grafted onto the backbone of either wild-type or COBRA HA polypeptides and methods of making and using the optimized HA polypeptides. There are five antigenic sites on the HA molecule of H1 and H3, which can be modified using the methods provided herein. For HA polypeptides of H1, the sites are referred to as Sa, Sb, Ca1, Ca2 and Cb. See, for example, Igarashi et al, PLOSOne, 5(1): e8553 (2010); Stray & Pittman, Virology Journal, 9:91 (2012); and Caton & Brownlee, Cell, 31(2 Pt 1):417-27 (1982), which are incorporated by reference herein in their entireties. By way of example, the Sa, Sb, Ca1, Ca2 and Cb antigenic sites of HA of the H1 subtype from A/PR/8/34 is shown below in Table 1.

TABLE 1
Antigenic Sites of HA from A/PuertoRico/8/34 (H1 subtype)

| Antigenic | |
|---|---|
| Site | Amino Acid Numbering Relative to SEQ ID NO: 11 |
| Sa | 124, 125, 128, 129, 158, 159, 161, 162, 163, |
| | 165, 166, 167, 247, 248 |
| Sb | 152, 155, 156, 159 188, 189, 192-198 |
| Ca1 | 165, 169, 173, 178, 203, 207, 212, 236, 240- |
| | 245, 270 |
| Ca2 | 132, 133, 136, 139-141, 143-145, 149, 220, |
| | 221, 224, 225 |
| Cb | 51, 70, 71, 73-75, 77-79, 87-91, 115, 117, |
| | 140, 255, 256, 259-266 |

For HA polypeptides of H3, the sites are referred to as A, B, C, D, and E. See, for example, Stray & Pittman, Virology Journal, 9:91 (2012); Smith et al, Science, 305(5682):371-376 (2004); and Chun-Chieh Shih, PNAS, 4(15):6283-8 (2007), which are incorporated by reference herein in their entireties. By way of example, the A, B, C, D, and E antigenic sites of HA of the H3 subtype from A/Hong Kong/1/1968 is shown below in Table 2.

TABLE 2
Antigenic Sites of HA from A/Hong Kong/1/1968 (H3 subtype)

| Antigenic | |
|---|---|
| Site | Amino Acid Numbering Relative to SEQ ID NO: 12 |
| A | 122-127, 129, 131-138, 140, 142-146 |
| B | 155-160, 163, 164, 186, 188-190, 192-199 |
| C | 49, 50, 53, 54, 271, 273, 275-278, 299, 307 |
| D | 167, 172-174, 201-207, 213-220, 222-227, |
| | 242, 244, 248 |
| E | 57, 62, 63, 67, 75, 78-83, 91, 92, 94, 260, |
| | 262 |

Using antigenic site sequences from HA molecules with neutralizing activity, these sequences can be grafted onto the backbone of wild-type or COBRA HA sequences to generate HA molecules with enhanced activity for eliciting broadly cross-reactive neutralizing antibodies. Thus, provided herein are isolated recombinant influenza HA molecules comprising modifications in one, two, three, four or five of the antigenic sites of an HA backbone sequence. Thus, the an HA backbone sequence can contain one or more of the Sa, Sb, Ca1, Ca2 or Cb antigenic sites of a H1 subtype HA molecule. Optionally, an HA backbone sequence can contain one or more of the A, B, C, D, or E antigenic sites of a H3 subtype HA molecule. Optionally, an HA backbone sequence can contain one or more of the BA, BB1, BB2, BC, BD or BE antigenic sites of a B subtype HA molecule. By way of example, the provided polypeptides comprise an HA backbone sequence with one or more antigenic sites from a different HA sequence. Thus, the provided polypeptides are hybrids of different sequences artificially combined to create new HA polypeptide sequences.

Hemagglutinin (HA) is an influenza virus surface glycoprotein. HA mediates binding of the virus particle to a host cells and subsequent entry of the virus into the host cell. The nucleotide and amino acid sequences of numerous influenza HA proteins are known in the art and are publically available, such as those deposited with GenBank. HA (along with NA) is one of the two major influenza virus antigenic determinants. As used herein, the term “HA backbone sequence,” refers to the amino acid or nucleic acid sequence of an HA molecule that is to be altered by modification of one or more antigenic sites on the backbone sequence. Exemplary HA backbone sequences include, but are not limited to, wild-type HA sequences, such as those deposited with GenBank, and COBRA HA sequences. Such sequences are described in, for example, U.S. Pat. No. 8,883,171, U.S. Publication No. 2014/0147459, and U.S. Publication No. 2015/0017196, which are incorporated by reference herein in their entireties. Specific examples of COBRA HA sequences include, but are not limited to, COBRA P-1 (SEQ ID NO:13), COBRA X-6 (SEQ ID NO:14), and COBRA X-3 (SEQ ID NO:15).

As used herein, the term “hybrid” refers to a molecule comprised of different elements or parts. By way of example, a hybrid amino acid sequence contains amino acid sequence of two different amino acid sequences. In the context of the present application, a hybrid HA polypeptide comprises the sequence of two different HA polypeptide sequences. By way of example, a hybrid HA polypeptide comprises an HA backbone and one or more antigenic sites from an HA molecule of a different sequence. The HA backbone can be of the same or a different subtype from the HA molecule of a different sequence. For example, the HA backbone can be from a first influenza isolate and the antigenic sites from a different influenza isolate. The isolates can be from the same or different subtypes.

A recombinant nucleic acid, protein or virus is one that has a sequence that is not naturally occurring and/or has a sequence that is made by an artificial combination of two otherwise separated segments of sequence. When used in reference to the optimized influenza HA polypeptides describe herein, for example, the term refers to the fact that the HA polypeptides have been designed (i.e., are not naturally occurring) and/or whose existence and production require one or more actions. The artificial production or combination is often accomplished by chemical synthesis and/or by the artificial manipulation of isolated segments of nucleic acids, for example, by genetic engineering techniques.

As used herein, an “isolated” biological component (such as a nucleic acid, protein or virus) has been substantially separated or purified away from other biological components (such as cell debris, or other proteins or nucleic acids). Biological components that have been “isolated” include those components purified by standard purification methods. The term also embraces recombinant nucleic acids, proteins or viruses, as well as chemically synthesized nucleic acids or peptides.

Exemplary recombinant, hybrid HA polypeptides include, but are not limited to, isolated influenza HA polypeptides comprising at least 95%, 96%, 97%, 98%, or 99% identity to SEQ ID NO:1; SEQ ID NO:2; or SEQ ID NO:3. Optionally, the isolated influenza HA polypeptides comprise at least 96% identity to SEQ ID NO:1, SEQ ID NO:2, or SEQ ID NO:3. Optionally, the isolated influenza HA polypeptides comprise at least 97% identity to SEQ ID NO:1, SEQ ID NO:2, or SEQ ID NO:3. Optionally, the isolated influenza HA polypeptides comprise at least 98% identity to SEQ ID NO:1, SEQ ID NO:2, or SEQ ID NO:3. Optionally, the isolated influenza HA polypeptides comprise at least 99% identity to SEQ ID NO:1, SEQ ID NO:2, or SEQ ID NO:3. Optionally, the isolated influenza HA polypeptides comprise SEQ ID NO:1; SEQ ID NO:2; or SEQ ID NO:3.

Provided herein are also methods of making the optimized influenza polypeptides. The methods can be used on any subtype of influenza HA molecule. Thus, provided is a method of making an optimized influenza virus polypeptide sequence comprising (i) obtaining the amino acid sequences of one or more polypeptides from one or more of influenza virus isolates, wherein the influenza virus isolates are from the same subtype, and (ii) modifying an influenza virus polypeptide backbone to include the one or more antigenic binding sites to make the optimized influenza virus polypeptide. Optionally, the method further includes determining the antigenic binding sites of the one or more polypeptides from the one or more influenza virus isolates of the same subtype, wherein the polypeptides comprise one or more antigenic binding sites Optionally, the influenza virus is selected from the group consisting of an H1, H3, H5, H7 or B influenza virus. Optionally, the polypeptide is a HA polypeptide. Optionally, the antigenic sites comprise Sa, Sb, Ca1, Ca2 or Cb antigenic sites from an H1 subtype HA molecule. Optionally, the antigenic sites comprise A, B, C, D, or E antigenic sites from an H3 subtype HA molecule. Optionally, the antigenic sites comprise BA, BB1, BB2, BC, BD or BE antigenic sites from a B subtype HA molecule. Thus, the optimized HA polypeptide can include any combination of these antigenic sites from an HA polypeptide with a sequence different from the HA polypeptide backbone sequence. Optionally, the optimized modified polypeptide comprises SEQ ID NO: 1; SEQ ID NO:2, or SEQ ID NO:3.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Also provided herein are isolated nucleic acid molecules comprising nucleotide sequences encoding the provided recombinant, hybrid influenza hemagglutinin (HA) polypeptides. Optionally, the polypeptides comprise a sequence that is at least 95% identical to SEQ ID NO:1; SEQ ID NO:2, or SEQ ID NO:3. Optionally, the polypeptides comprise SEQ ID NO: 1; SEQ ID NO:2, or SEQ ID NO:3. Optionally, the nucleic acid sequence comprise a sequence that is at least 95% identical to SEQ ID NO:4, SEQ ID NO:5, or SEQ ID NO:6. Optionally, the nucleic acid sequence comprises SEQ ID NO:4, SEQ ID NO:5, or SEQ ID NO:6.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments. the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides andAnalogues: A Practical Approach, Oxford University Press); and peptide nucleic acid backbones and linkages. Other analog nucleic acids include those with positive backbones; non-ionic backbones, and nonribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids.

Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

Nucleic acids are linear polymers (chains) of nucleotides, which consist of a purine or pyrimidine nucleobase or base, a pentose sugar, and a phosphate group. As used herein, a “polymer backbone” refers to the chain of pentose sugars and phosphate groups lacking the bases normally present in a nucleic acid.

Nucleic acids can include nonspecific sequences. As used herein, the term “nonspecific sequence” refers to a nucleic acid sequence that contains a series of residues that are not complementary to or are only partially complementary to any other nucleic acid sequence. By way of example, a nonspecific nucleic acid sequence is incapable of hybridizing to any other nucleic acid sequence under hybridizable conditions. Optionally, a nonspecific nucleic acid sequences is a sequence that is not substantially identical to any other nucleic acid sequence. By way of another example, a nonspecific nucleic acid sequence is a sequence of nucleic acid residues that does not function as an inhibitory nucleic acid. An “inhibitory nucleic acid” is a nucleic acid (e.g. DNA, RNA, polymer of nucleotide analogs) that is capable of binding to a target nucleic acid (e.g. an mRNA translatable into a protein) and reducing transcription of the target nucleic acid (e.g. mRNA from DNA) or reducing the translation of the target nucleic acid (e.g., mRNA) or altering transcript splicing (e.g. single stranded morpholino oligo).

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The terms “identical” or percent sequence “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. Employed algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

Also provided are vectors comprising nucleic acid sequences encoding the provided influenza hemagglutinin (HA) polypeptide. Optionally, the polypeptides comprise a sequence that is at least 95% identical to SEQ ID NO; SEQ ID NO:2, or SEQ ID NO:3. Optionally, the polypeptides comprise SEQ ID NO: 1; SEQ ID NO:2, or SEQ ID NO:3. Optionally, the nucleic acid sequences comprise a sequence that is at least 95% identical to SEQ ID NO:4, SEQ ID NO:5, or SEQ ID NO:6. Optionally, the nucleic acid sequences comprise SEQ ID NO:4, SEQ ID NO:5, or SEQ ID NO:6. Vectors comprising a nucleic acid sequence encoding an influenza neuraminidase (NA), a nucleic acid sequence encoding an HIV GAG polypeptide, and vectors comprising a nucleic acid sequence encoding an influenza M1 polypeptide, are also provided. Thus, provided are a plurality of vectors comprising (i) a vector comprising a nucleic acid sequence encoding an influenza hemagglutinin (HA) polypeptide selected from the group consisting of SEQ ID NO: 1; SEQ ID NO:2, and SEQ ID NO:3, (ii) a vector comprising a nucleic acid sequence encoding an influenza neuraminidase (NA), and (iii) a vector comprising a nucleic acid sequence encoding an HIV GAG polypeptide. Provided are a plurality of vectors comprising (i) a vector comprising a nucleic acid sequence encoding an influenza hemagglutinin (HA) polypeptide selected from the group consisting of SEQ ID NO:1; SEQ ID NO:2, or SEQ ID NO:3, (ii) a vector comprising a nucleic acid sequence encoding an influenza neuraminidase (NA), and (iii) a vector comprising a nucleic acid sequence encoding an influenza M1 polypeptide. Optionally, the amino acid sequence of the NA comprises SEQ ID NO:7 and the amino acid sequence of the M1 polypeptide comprises SEQ ID NO:9. Optionally, the nucleic acid sequence encoding the NA polypeptide comprises SEQ ID NO: 8 and the nucleic acid sequence encoding the M1 polypeptide comprises SEQ ID NO: 10.

As used herein, a matrix (M1) protein refers to the influenza virus structural protein found within the viral envelope. M1 is thought to function in assembly and budding. As used herein, a neuraminidase (NA) refers to the influenza virus membrane glycoprotein. NA is involved in the destruction of the cellular receptor for the viral HA by cleaving terminal sialic acid residues from carbohydrate moieties on the surfaces of infected cells. NA also cleaves sialic acid residues from viral proteins, preventing aggregation of viruses. NA (along with HA) is one of the two major influenza virus antigenic determinants.

As used herein, a vector refers to a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes. In some embodiments of the present disclosure, the vector encodes an influenza HA, NA or M1 protein. Optionally, the vector is the pTR600 expression vector (U.S. Patent Application Publication No. 2002/0106798; Ross et al., Nat. Immunol. 1(2):102-103, 2000; Green et al., Vaccine 20:242-248, 2001, which are incorporated by reference herein in their entireties).

Construction of suitable vectors containing the nucleic acid sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

The provided vectors can contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements. Enhancers can be used to function to increase transcription from nearby promoters. Vectors may also contain sequences necessary for the termination of transcription referred to as terminators. The identification and use of terminators in expression vectors is well established. Further, the vectors can include nucleic acid sequence encoding a marker product. This marker product is used to determine if the gene has been delivered to the cell and once delivered is being expressed.

Cells comprising the vectors and nucleic acids are provided. Such cells can be used to generate the provided recombinant, hybrid HA polypeptides and virus-like particles containing the recombinant, hybrid, HA polypeptides. Suitable cells include, but are not limited to, eukaryotic host cells, prokaryotic host cells, and mammalian host cells. For example, the cells can be yeast, insect, avian, plant, C. elegans, and mammalian host cells. Examples of mammalian cells include, but are not limited to, COS cells, baby hamster kidney cells, mouse L cells, LNCaP cells, Chinese hamster ovary (CHO) cells, human embryonic kidney (HEK) cells, African green monkey cells, CV1 cells, HeLa cells, MDCK cells, Vero, and Hep-2 cells. Prokaryotic host cells include bacterial cells, for example, E. coli, B. subtilis, and mycobacteria.

Provided are methods of making a virus like particle (VLP) comprising culturing the cells comprising the provided nucleic acids or vectors under conditions to produce the VLP and isolating the VLP. Provided is a method of making a VLP comprising transfecting a host cell with a vector encoding a HA polypeptide, a vector encoding an influenza NA polypeptide and a vector encoding an influenza M1 polypeptide under conditions sufficient to allow for expression of the HA, M1 and NA polypeptides and isolating the VLP. Provided is a method of making a VLP comprising transfecting a host cell with a vector encoding an HA polypeptide of, a vector encoding an influenza NA polypeptide and a vector encoding a HIV GAG polypeptide under conditions sufficient to allow for expression of the HA, HIV GAG and NA polypeptides and isolating the VLP. Optionally, the polypeptides comprise a sequence that is at least 95%, 96%, 97%, 98% or 99% identical to SEQ ID NO: 1 SEQ ID NO:2, or SEQ ID NO:3. Optionally, the polypeptides comprise a sequence that is at least 96% identical to SEQ ID NO:1 SEQ ID NO:2, or SEQ ID NO:3. Optionally, the polypeptides comprise a sequence that is at least 97% identical to SEQ ID NO:1 SEQ ID NO:2, or SEQ ID NO:3. Optionally, the polypeptides comprise a sequence that is at least 98% identical to SEQ ID NO: 1 SEQ ID NO:2, or SEQ ID NO:3. Optionally, the polypeptides comprise a sequence that is at least 99% identical to SEQ ID NO:1 SEQ ID NO:2, or SEQ ID NO:3. Optionally, the polypeptides comprise SEQ ID NO:1; SEQ ID NO:2, or SEQ ID NO:3. Optionally, the nucleic acid sequences comprise a sequence that is at least 95% identical to SEQ ID NO:4, SEQ ID NO:5, or SEQ ID NO:6. Optionally, the nucleic acid sequences comprise SEQ ID NO:4, SEQ ID NO:5, or SEQ ID NO:6. Optionally, the amino acid sequence of the NA comprises SEQ ID NO:7 and the amino acid sequence of the M1 polypeptide comprises SEQ ID NO:9. Optionally, the nucleic acid sequence encoding the NA polypeptide comprises SEQ ID NO:8 and the nucleic acid sequence encoding the M1 polypeptide comprises SEQ ID NO:10.

The influenza VLPs are made up of the HA, NA and M1 proteins. The production of influenza VLPs has been described in the art and is within the abilities of one of ordinary skill in the art. As described herein, influenza VLPs can be produced by transfection of host cells with plasmids encoding the HA, NA and M1 proteins. After incubation of the transfected cells for an appropriate time to allow for protein expression (such as for approximately 72 hours), VLPs can be isolated from cell culture supernatants. See, for example, U.S. Pat. No. 8,883,171, which is incorporated herein by reference in its entirety. The influenza VLPs disclosed herein can be used as influenza vaccines to elicit a protective immune response against H5N1 influenza viruses.

Provided are isolated virus-like particles comprising the provided HA polypeptides. Optionally, the virus-like particles comprise an influenza neuraminidase (NA) polypeptide and an influenza matrix (M1) polypeptide. Optionally, the amino acid sequence of the influenza NA polypeptide is at least 95% identical to SEQ ID NO:7, the amino acid sequence of the influenza M1 polypeptide is at least 95% identical to SEQ ID NO:9, or both. Optionally, the virus-like particles comprise an influenza neuraminidase (NA) polypeptide and an HIV GAG polypeptide.

As used herein, virus-like particles (VLP) refer to virus particles made up of one of more viral structural proteins, but lacking the viral genome. Because VLPs lack a viral genome, they are non-infectious. In addition, VLPs can often be produced by heterologous expression and can be easily purified. Most VLPs comprise at least a viral core protein that drives budding and release of particles from a host cell. One example of such a core protein is influenza M1. In some embodiments herein, an influenza VLP comprises the HA, NA and M1 proteins. As described herein, influenza VLPs can be produced by transfection of host cells with plasmids encoding the HA, NA and M1 proteins. After incubation of the transfected cells for an appropriate time to allow for protein expression (such as for approximately 72 hours), VLPs can be isolated from cell culture supernatants. Example 1 provides an exemplary protocol for purifying influenza VLPs from cell supernatants. In this example, VLPs are isolated by low speed centrifugation (to remove cell debris), vacuum filtration and ultracentrifugation through 20% glycerol.

Optionally, recombinant whole influenza viruses comprising the herein provided VIPER HA polypeptides are produced. Recombinant whole influenza viruses can be produced by plasmid-based reverse genetics and cell-based or egg-based technologies. Recombinant viruses containing internal protein genes from a donor virus may be used to prepare inactivated influenza virus vaccines (see, e.g., Fodor, E. et al. Rescue of influenza A virus from Recombinant DNA. J. Virol., 1999, 73, 9679-9682; incorporated by reference herein). Recombinant whole influenza viruses can be used to elicit protective immune responses against influenza viruses; for example, they can be administered as components of a live-attenuated or split-inactivated vaccine.

Thus, the immunogenic compositions and vaccines described herein may comprise one of three types of antigen preparation: inactivated whole virus, sub-virions where purified virus particles are disrupted with detergents or other reagents to solubilize the lipid envelope (“split” vaccine) or purified structural influenza polypeptide (“subunit” vaccine). Optionally, virus can be inactivated by treatment with formaldehyde, beta-propiolactone, ether, ether with detergent (such as TWEEN-80®), cetyl trimethyl ammonium bromide (CTAB) and Triton N101, sodium deoxycholate and tri(n-butyl) phosphate. Inactivation can occur after or prior to clarification of allantoic fluid (from virus produced in eggs); the virions are isolated and purified by centrifugation (Nicholson et al., eds., 1998, Textbook of Influenza, Blackwell Science, Malden, Mass.; incorporated herein by reference). To assess the potency of the vaccine, the single radial immunodiffusion (SRD) test can be used (Schild et al., 1975, Bull. World Health Organ., 52:43-50 & 223-31; Mostow et al., 1975, J. Clin. Microbiol., 2:531; both of which are incorporated herein by reference).

Optionally, influenza virus for use in vaccines is grown in eggs, for example, in embryonated hen eggs, in which case the harvested material is allantoic fluid. Alternatively or additionally, influenza virus and/or the provided influenza hemagglutinin (HA) polypeptide may be produced from any method using tissue culture to grow the virus. Suitable cell substrates for growing the virus or otherwise recombinantly producing the engineered, structural influenza polypeptides include, for example, CHO cells, dog kidney cells such as MDCK or cells from a clone of MDCK, MDCK-like cells, monkey kidney cells such as AGMK cells including Vero cells, cultured epithelial cells as continuous cell lines, 293T cells, BK-21 cells, CV-1 cells, or any other mammalian cell type suitable for the production of influenza virus (including upper airway epithelial cells) for vaccine purposes, readily available from commercial sources (e.g., ATCC, Rockville, Md.). Suitable cell substrates also include human cells such as MRC-5 cells. Suitable cell substrates are not limited to cell lines; for example primary cells such as chicken embryo fibroblasts are also included.

Provided are compositions comprising one or more of the inactivated virus, virus-like particles or one or more of the HA polypeptides. Optionally, the compositions comprise a pharmaceutically acceptable excipient or pharmaceutically acceptable carrier. Suitable carriers and their formulations are described in Remington: The Science and Practice of Pharmacy, 22nd Edition, Loyd V. Allen et al., editors, Pharmaceutical Press (2012). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

Optionally, a pharmaceutical composition can also contain a pharmaceutically acceptable carrier or adjuvant for administration of the vaccine, e.g., the HA polypeptides or VLPs. Optionally, the carrier is pharmaceutically acceptable for use in humans. The carrier or adjuvant should not itself induce the production of antibodies harmful to the individual receiving the composition and should not be toxic. Suitable carriers can be large, slowly metabolized macromolecules such as proteins, polypeptides, liposomes, polysaccharides, polylactic acids, polyglycolic acids, polymeric amino acids, ammo acid copolymers and inactive virus particles.

An adjuvant refers to a substance or vehicle that non-specifically enhances the immune response to an antigen. Adjuvants can include a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL. Optionally, the adjuvant is a squalene-based adjuvant. Squalene-based adjuvants are known and include, but are not limited to, MF59 and AS03.

Pharmaceutically acceptable carriers in therapeutic compositions can additionally contain liquids such as water, saline, glycerol and ethanol. Additionally, auxiliary substances, such as wetting or emulsifying agents or pH buffering substances, can be present in such compositions. Such carriers enable the pharmaceutical compositions to be formulated as tablets, pills, dragees, capsules, liquids, gels, syrups, slurries and suspensions, for ingestion by the patient.

The compositions of the presently disclosed subject matter can further comprise a carrier to facilitate composition preparation and administration. Any suitable delivery vehicle or carrier can be used, including but not limited to a microcapsule, for example a microsphere or a nanosphere (Manome et al. (1994) Cancer Res 54:5408-5413; Saltzman & Fung (1997) Adv Drug Deliv Rev 26:209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al. (1997) Cancer Res 57:1447-1451 and U.S. Pat. Nos. 4,551,482, 5,714,166, 5,510,103, 5,490,840, and 5,855,900), and a polysome (U.S. Pat. No. 5,922,545).

Suitable formulations for the provided compositions can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of in some embodiments 0.1 to 10 mg/ml, in some embodiments about 2.0 mg/ml; and/or mannitol or another sugar in the range of in some embodiments 10 to 100 mg/ml, in some embodiments about 30 mg/ml; and/or phosphate-buffered saline (PBS). Any other agents conventional in the art having regard to the type of formulation in question can be used. In some embodiments, the carrier is pharmaceutically acceptable. In some embodiments the carrier is pharmaceutically acceptable for use in humans.

Pharmaceutical compositions of the present disclosure can have a pH between 5.5 and 8.5, between 6 and 8, or about 7. Optionally, the pH can be maintained by the use of a buffer. The composition can be sterile and/or pyrogen free. The composition can be isotonic with respect to humans. Pharmaceutical compositions of the presently disclosed subject matter can be supplied in hermetically-sealed containers.

Pharmaceutical compositions can include an effective amount of one or more HA polypeptides and/or VLPs as described herein. Optionally, a pharmaceutical composition can comprise an amount that is sufficient to treat, ameliorate, or prevent a desired disease or condition, or to exhibit a detectable therapeutic effect. Therapeutic effects also include reduction in physical symptoms. The precise effective amount for any particular subject will depend upon their size and health, the nature and extent of the condition, and therapeutics or combination of therapeutics selected for administration. The effective amount for a given situation is determined by routine experimentation as practiced by one of ordinary skill in the art.

The pharmaceutical preparation is preferably in unit dosage form. In such form the preparation is subdivided into unit doses containing appropriate quantities of the active component. Thus, the pharmaceutical compositions can be administered in a variety of unit dosage forms depending upon the method of administration. For example, unit dosage forms suitable for oral administration include, but are not limited to, powder, tablets, pills, capsules and lozenges.

Provided are methods of eliciting an immune response to influenza virus in a subject, comprising administering the influenza HA polypeptide, wherein the administering elicits an immune response to influenza virus. Optionally, the HA polypeptides are administered as inactivated virus or virus-like particles. Optionally, the methods include administering an adjuvant to the subject. Optionally, the administering comprises administering to the subject a first influenza HA polypeptide having an amino acid sequence selected from the group consisting of SEQ ID NO: 1; SEQ ID NO:2; or SEQ ID NO:3, and wherein the method further comprising administering to the subject a second influenza HA polypeptide having an amino acid sequence different from the first influenza HA polypeptide. Optionally, the first and second influenza HA polypeptides are administered simultaneously or concurrently. By way of example, the methods can include administering to the subject an influenza HA polypeptide having an amino acid sequence comprising SEQ ID NO: 1 and administering to the subject after a suitable period of time an influenza HA polypeptide having an amino acid sequence comprising SEQ ID NO:2.

Provided are methods of immunizing a subject against influenza virus, comprising administering to the subject a composition comprising a VLP or HA polypeptide, wherein the administering immunizes the subject against the influenza virus. Optionally, the HA polypeptides are administered as inactivated virus or virus-like particles. Also provided are methods of treating an influenza virus infection comprising administering to the subject a composition comprising a VLP or HA polypeptide, wherein the administering treats the infection. Optionally, the composition further comprises an adjuvant. Optionally, the composition is administered intramuscularly. Optionally, the composition comprises about 1 to about 25 μg of the VLP. Optionally, the composition comprises about 1 to 3 μg of the VLP. Optionally, the composition comprises about 3-15 μg of the VLP.

As used herein, vaccine refers to a preparation of immunogenic material capable of stimulating an immune response, administered for the prevention, amelioration, or treatment of disease, such as an infectious disease. The immunogenic material may include, for example, attenuated or killed microorganisms (such as attenuated viruses), or antigenic proteins, peptides or DNA derived from them. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous or intramuscular. Vaccines may be administered with an adjuvant to boost the immune response.

As used herein, an immune response refers to a response of a cell of the immune system, such as a B-cell, T-cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like. In some embodiments, methods of measuring an immune response include hemagglutination inhibition (HAI) assays to assess functional antibody binding to the HA polypeptides described herein, thereby serving as surrogate measure of influenza vaccine efficacy. HAI assays may use chicken, turkey or horse erythrocytes for the detection of antibodies specific for H3N2. Protective immune responses can be demonstrated by eliciting an average HAI titer of greater than 1:40, which has been correlated with prevention and reduction of influenza illness. HAI antibody titers of approximately 1:32 to 1:40 will generally protect about 50% of subjects from infection after immunization with inactivated human influenza virus vaccine. See, e.g., Treanor, J. and Wright, P. F. Immune correlates of protection against influenza in the human challenge model. Dev. Biol. (Basel), 2003, 115:97-104, which is incorporated by reference herein in its entirety. Optionally, elicitation of a protective immune response can by identified by seroconversion rates. A protective level of seroconversion may be defined as at least a 4-fold rise in HAI titer, for example, a pre-administration or vaccination HAI titer of less than 1:10 and a post vaccinate titer of greater than or equal to 1:40. In other words, successful rates of seroconversion may be defined as the percentage of subjects with either a pre-vaccination HAI titer less than about 1:10 and a post-vaccination HAI titer of greater than about 1:40 or a pre-vaccination HAI titer greater than about 1:10 and a minimum four-fold rise in post-vaccination HAI antibody titer.

As used herein, an immunogen refers to a compound, composition, or substance which is capable, under appropriate conditions, of stimulating an immune response, such as the production of antibodies or a T-cell response in an animal, including compositions that are injected or absorbed into an animal. As used herein, as “immunogenic composition” is a composition comprising an immunogen (such as an HA polypeptide).

As used herein, the term “antigen” refers to a substance that prompts the generation of antibodies and can cause an immune response. It can be used interchangeably in the present disclosure with the term “immunogen”. In the strict sense, immunogens are those substances that elicit a response from the immune system, whereas antigens are defined as substances that bind to specific antibodies. An antigen or fragment thereof can be a molecule (i.e., an epitope) that makes contact with a particular antibody. When a protein or a fragment of a protein is used to immunize a host animal, numerous regions of the protein can induce the production of antibodies (i.e., elicit the immune response), which bind specifically to the antigen (given regions or three-dimensional structures on the protein).

As used herein, immunize refers to rendering a subject protected from an infectious disease, such as by vaccination.

As used herein, administering a composition to a subject means to give, apply or bring the composition into contact with the subject. Administration can be accomplished by any of a number of routes, such as, for example, topical, oral, subcutaneous, intramuscular, intraperitoneal, intravenous, intrathecal and intramuscular.

Influenza HA polypeptides and VLPs comprising HA polypeptides, or compositions thereof, can be administered to a subject by any of the routes normally used for introducing recombinant virus into a subject. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local.

Influenza VLPs, or compositions thereof, are administered in any suitable manner, such as with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent influenza virus infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

The pharmaceutical compositions described herein can be administered in a variety of unit dosage forms depending upon the method of administration. Dosages for typical antibody pharmaceutical compositions are well known to those of skill in the art. Such dosages are typically advisory in nature and are adjusted depending on the particular therapeutic context or patient tolerance. The amount antibody adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

“Therapeutically-effective amount” or “an amount effective to reduce or eliminate infection” or “an effective amount” refers to an amount of an antibody composition that is sufficient to prevent influenza virus infection or to alleviate (e.g., mitigate, decrease, reduce) at least one of the symptoms associated with such an infection. It is not necessary that the administration of the composition eliminate the symptoms of influenza virus infection, as long as the benefits of administration of the composition outweigh the detriments. Likewise, the terms “treat” and “treating” in reference to influenza virus infection, as used herein, are not intended to mean that the subject is necessarily cured of infection or that all clinical signs thereof are eliminated, only that some alleviation or improvement in the condition of the subject is effected by administration of the composition.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. In some embodiments, the terms treatment, treat or treating refer to methods of preventing the establishment of, or development of one or more consequences or symptoms associated with, infection by influenza A virus subtype, including fever, myalgia, headache, malaise, nonproductive cough, sore throat, rhinitis, weight loss, otitis media, nausea, vomiting, and death. For example, the terms treatment, treat, or treating can refer to methods of reducing the effects of one or more consequences or symptoms of infection by influenza A virus subtype. Thus, the methods of treatment disclosed herein can refer to a 10%, 20%, 30%, 40%, 50%, 60%, 70%, 80%, 90%, or 100% reduction in the severity of one or more consequences or symptoms of infection by influenza A virus subtype, including fever, myalgia, headache, malaise, nonproductive cough, sore throat, rhinitis, weight loss, otitis media, nausea, vomiting, and death. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination. Optionally, the terms treatment, treat or treating refer to methods of shortening the duration of one or more symptoms of infection by influenza A virus subtype H3N2, including fever, myalgia, headache, malaise, nonproductive cough, sore throat, rhinitis, weight loss, otitis media, nausea, vomiting. In certain embodiments, the duration of symptoms is shortened to less than 2 weeks, to less than 7 days, and/or to less than 3 days.

Vertebrate,” “mammal,” “subject,” “mammalian subject,” or “patient” are used interchangeably and refer to mammals such as human patients and non-human primates, as well as experimental animals such as rabbits, rats, and mice, cows, horses, goats, and other animals. Animals include all vertebrates, e.g., mammals and non-mammals, such as mice, sheep, dogs, cows, avian species, ducks, geese, pigs, chickens, amphibians, and reptiles.

A “control” or “standard control” refers to a sample, measurement, or value that serves as a reference, usually a known reference, for comparison to a test sample, measurement, or value. For example, a test sample can be taken from a patient suspected of having a given disease (e.g. an autoimmune disease, inflammatory autoimmune disease, cancer, infectious disease, immune disease, or other disease) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

Also provided are kits comprising the HA polypeptides produced in accordance with the present disclosure which can be used, for instance, for therapeutic applications described above. Optionally, the kit comprises inactivated viruses of VLPs comprising the HA polypeptides. The article of manufacture comprises a container with a label. Suitable containers include, for example, bottles, vials, and test tubes. The containers can be formed from a variety of materials such as glass or plastic. The container holds a composition which includes an active agent that is effective for therapeutic applications, such as described above. The active agent in the composition can comprise the HA polypeptides. The label on the container indicates that the composition is used for a particular therapy or non-therapeutic application, and can also indicate directions for either in vivo or in vitro use, such as those described above.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the claims.

### EXAMPLE

### Example 1. Vaccines Intelligently Produced by Epitope Recombination (VIPER) for Influenza

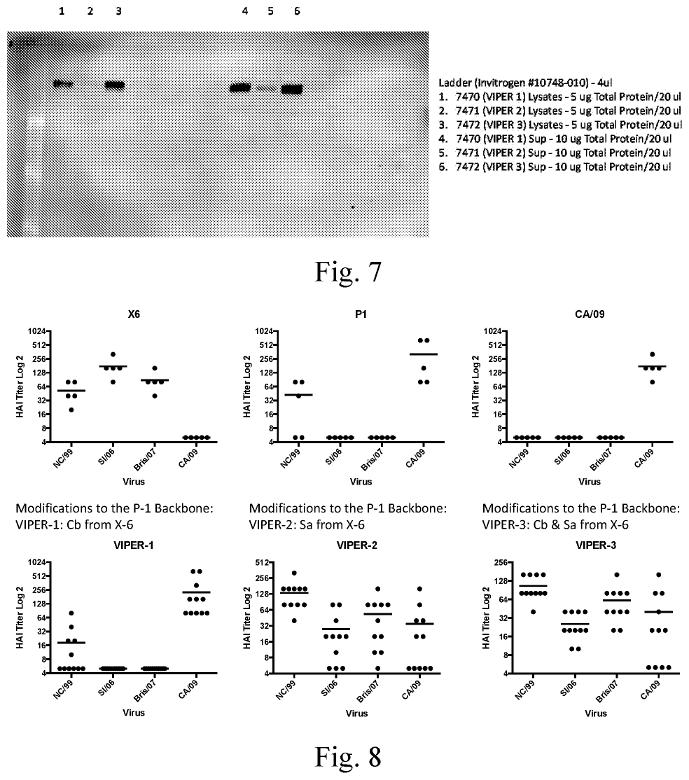

VIPER sequences were designed using COBRA P-1 (SEQ ID NO:13), and COBRA X-6 (SEQ ID NO: 14) sequences. After analysis of COBRA P-1, and COBRA X-6 sequences differences in the SA antigenic site and Cb antigenic site were observed. These differences were compared to wild type sequences that were distinctly recognized by only one of the two COBRA vaccines. Specifically, the sequences in A/Brisbane/59/07 (recognized in serum from X-6 vaccinated animals) and A/California/07/09 (recognized by P-1 vaccinated animals). Using the P-1 sequence as the “backbone” sequence, site changes were made to the Sa and/or Cb site to incorporate the X-6 sequences at these sites that were homologous with the wild-type A/Brisbane/59/07. The overall goal to broaden the HAI response that P-1 elicits by also capturing the A/Brisbane59/07. To do this three VIPER sequences were made. The VIPER 1 sequence (SEQ ID NO: 1) shows a change in the COBRA P-1 backbone starting at amino acid number 87, the P-1 sequence was changed from LLSAR to LISKE at the Cb antigenic site. The VIPER 2 sequence (SEQ ID NO:2) shows a change in the COBRA P-1 backbone starting at amino acid number 144, the P-1 sequence was changed from NTTK to TV-T at the Sa antigenic site. The VIPER 3 sequence (SEQ ID NO:3) has both the changes from VIPER 1 and VIPER 2, i.e., at the Sa and Cb antigenic sites. FIG. 7 is an image of a Western blot showing the detection of VIPER1, VIPER2 and VIPER3 HA on the surface of VLPs following purification.

It was hypothesized that VIPERs will expand the breadth of the P-1 vaccine to include Bris/07. To test this hypothesis, an experimental design was developed as shown in Table 3. Mice were vaccinated with each VLP expressing one of the VIPER H1 HA gene products at week 0, 4, and 8. These results were compared to mice vaccinated with unmodified “backbone” X6 and P1 COBRA VLPs and wild-type A/California/07/09 (“CA/09”). Serum was collected at week 8 and tested against a small panel of H1N1 viruses in an HAI assay. Seasonal strain viruses in the panel are represented by A/New Caledonia/20/1999 (“NC/99”), A/Solomon Islands/03/2006 (“SI/06) and A/Brisbane/59/07 (“Bris/07”). The pandemic strain in the panel was A/California/07/09 (CA/09).

TABLE 3
Experimental Design to Evaluate VIPER VLPs.

| | | | | | | |
|---|---|---|---|---|---|---|
| | | Week 0 | Week 4 | Week 8 | | |
| | Mice | Prime | Boost | Boost | Week 12 | |
| Groups | (n) | VLP | VLP | VLP | Challenge | Dose |

| | | | | |
|---|---|---|---|---|
| 1 | 11 | VIPER 1 | CA/09 | 3 μg |
| 2 | 11 | VIPER 2 | CA/09 | 3 μg |
| 3 | 11 | VIPER 3 | CA/09 | 3 μg |
| 4 | 5 | CA/09 | CA/09 | 3 μg |
| 5 | 5 | P1 | CA/09 | 3 μg |
| 6 | 5 | X6 | CA/09 | 3 μg |
| 7 | 11 | Mock | CA/09 | 3 μg |

Bleeds Schedule: Week 8 & Week 12; Adjuvanted w/AFO3;

The HAI assays demonstrated that the H1 VIPER HAs demonstrated a broader neutralizing antibody response against both seasonal and pandemic H1N1 viruses (FIG. 8) compared to the “backbone” X6 and P1 COBRA VLPs and CA/09 controls (top panel of slide 2). VIPER2 and VIPER3 demonstrated a broader HAI response by also capturing Bris/07.

1. A recombinant influenza HA polypeptide comprising at least 95% identity to SEQ ID NO:1; SEQ IS NO:2; or SEQ ID NO:3.

2. The recombinant influenza HA polypeptide of claim 1 comprising SEQ ID NO:1; SEQ IS NO:2; or SEQ ID NO:3.

3. (canceled)

4. A method of eliciting an immune response to influenza virus in a subject, comprising administering an influenza HA polypeptide comprising at least 95% identity to SEQ ID NO:1; SEQ IS NO:2; or SEQ ID NO:3, wherein the administering elicits an immune response to influenza virus.

5. The method of claim 4, further comprising administering an adjuvant to the subject.

6. The method of claim 4 wherein the administering comprises administering to the subject a first influenza HA polypeptide having an amino acid sequence selected from the group consisting of SEQ ID NO:1; SEQ IS NO:2; or SEQ ID NO:3, and wherein the method further comprising administering to the subject a second influenza HA polypeptide having an amino acid sequence different from the first influenza HA polypeptide.

7. The method of claim 6, wherein the first and second influenza HA polypeptides are administered simultaneously or concurrently.

8. A virus-like particle comprising a polypeptide comprising at least 95% identity to SEQ ID NO:1; SEQ IS NO:2; or SEQ ID NO:3.

9. The virus-like particle of claim 8, further comprising an influenza neuraminidase (NA) polypeptide and an influenza matrix (M1) polypeptide.

10. The virus-like particle of claim 9, wherein the amino acid sequence of the influenza NA polypeptide is at least 95% identical to SEQ ID NO:7, the amino acid sequence of the influenza M1 polypeptide is at least 95% identical to SEQ ID NO:9, or both.

11. The virus-like particle of claim 8, further comprising an influenza neuraminidase (NA) polypeptide and an HIV GAG polypeptide.

12-29. (canceled)

30. The method of claim 4 of eliciting an immune response to influenza virus in a subject, wherein the influenza HA polypeptide comprises SEQ ID NO:1; SEQ IS NO:2; or SEQ ID NO:3.

31. The virus-like particle of claim 8, wherein the polypeptide comprises SEQ ID NO:1; SEQ IS NO:2; or SEQ ID NO:3.

32. The virus-like particle of claim 9, wherein the polypeptide comprises SEQ ID NO:1; SEQ IS NO:2; or SEQ ID NO:3.

33. The virus-like particle of claim 10, wherein the polypeptide comprises SEQ ID NO:1; SEQ IS NO:2; or SEQ ID NO:3.

34. The virus-like particle of claim 9, wherein the amino acid sequence of the influenza NA polypeptide is SEQ ID NO:7, the amino acid sequence of the influenza M1 polypeptide is SEQ ID NO:9, or both.

35. The virus-like particle of claim 11, wherein the polypeptide comprises SEQ ID NO:1; SEQ IS NO:2; or SEQ ID NO:3.

36. The virus-like particle of claim 11, further comprising an influenza neuraminidase (NA) polypeptide and an influenza matrix (M1) polypeptide.

37. The virus-like particle of claim 11, wherein the amino acid sequence of the influenza NA polypeptide is at least 95% identical to SEQ ID NO:7, the amino acid sequence of the influenza M1 polypeptide is at least 95% identical to SEQ ID NO:9, or both.
